(19) United States
(12) Patent Application Publication
(10) Pub. No.: US 2020/0254152 A1
(43) Pub. Date: Aug. 13, 2020

(54) COATING SYSTEM FOR MEDICAL DEVICES

(71) Applicant: RONTIS HELLAS S.A., ATHENS (GR)

(72) Inventor: ANARGYROS MOULAS, LARISA (GR)

(73) Assignee: RONTIS HELLAS S.A., ATHENS (GR)

(21) Appl. No.: 16/270,686

(22) Filed: Feb. 8, 2019

(51) Int. Cl. A61L 31/10; A61L 27/34; A61L 31/16; A61L 27/54

(52) U.S. Cl. CPC A61L 31/10; A61L 27/34; A61L 31/16; A61L 27/54; A61L 2420/02; A61L 2300/258; A61L 2300/404; A61L 2300/408; A61L 2300/42; A61L 2300/252; A61L 2300/416

(57) ABSTRACT

The present invention relates to a coating system for medical devices comprising at least one cellulose compound or derivative thereof alone or in combination with other substances. The coating system may further comprise at least one active ingredient intended to treat a disease and the coating has favorable drug eluting properties. The present invention has applications in implantable or non-implantable medical devices such as balloons, stents, grafts, patches and implants of all kinds.

### FIELD OF THE INVENTION

The present invention relates to an improved coating system for medical devices such as catheters, balloons, stents, grafts, patches and implants. The coating system includes a medical device and a coating that is applied on a surface of the device. The coating comprises at least one cellulose compound or derivative thereof alone or combined with at least one pharmaceutical ingredient. The invention also relates to a method for the preparation of the devices.

### BACKGROUND OF THE INVENTION

Medical devices are coated for various reasons, such as biocompatibility, hydrophilicity, lubricity, functionality and protection of the device from contact with body tissues or fluids. Additionally coatings on medical devices also serve as drug delivery systems.

Several types of coatings for medical devices exist with different physical and chemical properties depending on the application. The prior art corresponds partially to the needs of coating of medical devices but defects exist and there is a need for improvement with regard to biocompatibility, flexibility, durability and chemical compatibility of the coatings with the medical device made of various materials. More specifically there is a need for flexible, biocompatible coatings applicable to medical devices made of various different materials such as metals and polymers.

Moreover, the drug delivery systems for medical devices available on the market and particularly those used in endovascular devices such as balloons, stents, patches and grafts, include a carrier, usually polymeric, in which the drug is incorporated or dissolved or encapsulated. Depending on the application, for example balloon or stent, the drug delivery systems are required to have certain release characteristics and particularly certain release profile according to the specific application. As an example, drug delivery systems for stents should have sustained release profile for a relatively longer period of days or weeks or months, while in the case of drug coated balloons, the drug must be eluted within seconds or minutes. Other devices may have different requirements.

Currently known polymeric coating systems partially fulfill these requirements. However, the prior art has encountered substantial difficulties in the development of coating systems that can be simultaneously biocompatible, with favorable mechanical properties, and have adjustable drug release profile. Moreover, it is desired that the coating systems be made with sustainable raw materials.

Existing coatings for medical devices only partially fulfill the existing requirements. Most existing coating materials do not originate from sustainable renewable sources while the biocompatibility and mechanical properties of existing coatings are not always optimal.

Various drug-eluting stents are already known to release antiproliferative agents at the site of arterial injury to attenuate neointimal formation and treat effectively arterial disease. These drug-eluting stents typically comprise a metallic stent, a polymer-based drug delivery platform, and a pharmacologic agent such as an immunosuppressant and/or antiproliferative compound. However, there is a need for improvement in the biocompatibility of coatings, adjustability of drug eluting profile, compatibility of drug eluting system with different drugs and use of sustainable materials.

Furthermore, drug-eluting balloons (DEB) or Drug Coated Balloons (DCB) are also known medical devices used in angioplasty. These balloons typically comprise an angioplasty balloon covered with an active pharmaceutical ingredient (drug) and a drug release system. The active ingredient used on DEB is usually a lipophilic drug, commonly paclitaxel or rapamycin and its derivatives and is released to the vessel wall during inflation of the balloon. The whole system is designed in order to have a high absorption rate through the vessel wall to compensate for the short period of contact between the inflated balloon and the vessel wall itself, and to maintain a sustained effect once released. However, existing coatings on the DEBs are not very stable and can be easily eroded. During transition to the site of stenosis through the circulation system, the balloon is exposed to the blood stream and the drug is partially washed out so that only a small part of the total drug loaded on a balloon, usually less than 10%, is delivered to the artery and the other part of the loaded drug is lost in the circulation, raising concerns about possible systemic effects of the drug and about the consistency and repeatability of the drug delivery process.

Hence, there still exists a need for improving the coating systems for medical devices so as to have less loss of drug in the circulation, better biocompatibility, adjustable favorable mechanical properties, such as elasticity and durability and better protection of the device from contact with the tissues, adjustable drug release profile and compatibility with a variety of drugs.

### SUMMARY OF THE INVENTION

It is, therefore, an object of the present invention to provide an improved biocompatible coating system for medical devices, by using sustainable materials derived from natural sources with better biocompatibility with the artery wall and favorable mechanical, biochemical and drug release properties that overcomes the deficiencies of the prior art.

Moreover, it is another object of the present invention to provide a coating system for a DEB, overcoming the deficiencies of existing DEBs which have reduced biocompatibility, reduced elasticity, reduced device protection due to mechanical behaviour, reduced adjustability of the drug release rate, reduced drug delivery capacity and excessive drug loss in the circulation.

A further aspect of the present invention is to employ a suitable method for the preparation of a coating system for medical devices that is convenient, efficient, and cost effective.

In accordance with the above objects of the present invention, a coating system for medical devices is provided, comprising at least one cellulose compound or derivative thereof.

According to another embodiment of the present invention, a process for the preparation of a coating system for medical devices, which comprises a process for the preparation of a coating system for medical devices for the prevention and/or treatment of restenosis and neointimal formation during or after vascular procedures and/or implantation of implants comprising an implantable and non-implantable device, such as stent, balloon or graft, is provided wherein said coating system comprises at least one cellulose compound or derivative thereof, and a therapeutically effective amount of at least one active ingredient, said process comprises the following steps:
Preparation of a first mixture of at least one cellulose compound and dilution or dispersion of said cellulose compound in an appropriate solvent system;
preparation of a second mixture of an active ingredient in an appropriate solvent system;
Application of the first and second mixtures on the surface of the medical device such as a stent, balloon or graft with a suitable method, selected from dipping, immersion, spraying, preferably microspraying, micropipetting, casting in one or more steps, in a single or successive layers; and
Removal of possible remaining solvents with evaporation

Further preferred embodiments of the present invention are defined in dependent claims 2 to 10.

Other objects and advantages of the present invention will become apparent to those skilled in the art in view of the following detailed description and claims.

### DETAILED DESCRIPTION OF THE INVENTION

It has been surprisingly found that the object of the present invention is achieved by employing a coating system comprising at least one cellulose compound or derivative thereof. The cellulose compounds are biocompatible and possess favorable mechanical, chemical and biochemical properties as well as favorable and adjustable drug release properties. With the use of the cellulose compounds, biocompatible coatings for medical devices with desirable physical and release properties can be prepared.

Cellulose is the most abundant naturally occurring organic compound and thus the most abundant polymer on earth. It serves as a structural component of the cell wall of green plants, many forms of algae and oomycetes. Thus cellulose is a sustainable raw material.

Chemically, cellulose is a naturally occurring polymer of glucose. It is a linear and fairly rigid homopolymer consisting of D-anhydroglucopyranose (glucose) monomeric units linked with a β (1→4) glycosidic bond (a covalent bond) formed between C-1 and C-4 of adjacent glucose moieties. The chemical formula of cellulose is (C₆H₁₀O₅)ₙ, whereas n is usually a large number in the order of hundreds to thousands. The chain length of the polymer may be different depending on the plant origin of the cellulose source and the naturally occurring celluloses have an n value around 10000. Technical grades of cellulose have an n value of 800-3000 and microcrystalline cellulose an n value of 300-600.

In contrast with polymer and other substances originating from the petrochemical industry, most cellulose compounds are sustainable raw materials derived from natural sources.

For the purposes of the present invention, the term “cellulose compound” refers to cellulose and all its derivatives. More specifically the term “cellulose compound” refers initially to all forms of cellulose including but not limited to microcrystalline cellulose, hydrolyzed and modified celluloses of various grades and molecular weights.

Cellulose compounds, which can be used individually or in combination in the present invention, may be selected, but not limited, from the following: Cellulose ether derivatives of various molecular weights including but not limited to Methyl cellulose (MC), Ethyl cellulose (EC), Hydroxyethyl cellulose (HEC), ethylhydroxyethyl cellulose, (EHEC), Hydroxypropyl cellulose (HPC), hydroxypropylmethyl cellulose (HPMC), hydroxypropylmethyl cellulose phthalate, benzyl cellulose, carboxymethyl cellulose (CMC) and sodium carboxymethyl cellulose (NaCMC); Cellulose ester derivatives of various molecular weights including but not limited to cellulose acetate (CA), cellulose triacetate, cellulose acetate phthalate (CAP), Cellulose acetate butyrate (CAB), Cellulose acetate trimelitate (CAT), hydroxypropylmethyl cellulose phthalate (HPMCP), cellulose nitrate, cellulose sulphate; cellulose acetals such as but not limited to formaldehyde and acetaldehyde acetals; aminocellulose; cationic cellulose derivatives; any other type of cellulose or cellulose derivative.

The coating system according to the present invention can be used alone for purposes such as surface properties modification, biocompatibility enhancement and device protection from contact with body fluids or tissues.

Moreover, the coating system for medical devices of the present invention may also comprise at least one active pharmaceutical ingredient for the prevention and/or treatment of vascular diseases and/or restenosis, wherein said coating system is locally applied on the medical device.

It has been found that coating systems for medical devices made with the use of cellulose compounds are very biocompatible and additionally are compatible and mixable with a variety of active pharmaceutical substances that can be used for treatment of artery disease and for reducing the rate of possible restenosis and thrombosis. Additionally, several cellulose compounds and their mixtures are soluble in a variety of solvents from very polar such as water to non-polar such as chloroform, thus enabling effective mixture of the coating with active pharmaceutical substances of different polarity and chemical nature.

Compatibility and ability to mix with drugs of various levels of hydrophilicity—hydrophobicity along with the adjustability of the release profile, is an important advantage of the cellulose compounds according to the present invention.

Moreover, it has also been found that the cellulose compounds have good mechanical properties and film forming ability and can offer an adjustable protection from degradation for stents made of biodegradable metals, such as magnesium or biodegradable polymers, such as polylactide, polyglycolide, polycaprolactone etc. and their mixtures. The latter is particularly important when the degradation time of a biodegradable device needs to be prolonged.

An important advantage of coatings made with the use of cellulose compounds is the ability to adjust the drug release profile. This can be achieved by using different types of cellulose compounds alone or in combination with each other or with other excipients, thus allowing limitless combinations with different properties that enable the precise adjustment of the drug release profile as desired.

Additionally, it was found that in the case of drug eluting balloons, cellulose compounds used as coatings have the property to deliver sufficient amount of drugs to the artery wall with less loss into the circulation. Most existing drug eluting balloons lose as much as 90-95% of the drug in the circulation while balloons comprising coatings with cellulose compounds typically do not lose more than 50-80% of the drug in the circulation, while delivering similar amounts of drug to the artery wall. This property of the present invention is advantageous since it can reduce possible systemic effects of the drugs used in drug eluting balloons.

The coating system for medical devices according to the present invention comprising at least one cellulose compound and at least one active pharmaceutical ingredient is used for the prevention and/or treatment of vascular disease and restenosis.

The active ingredients can be applied locally on appropriate devices such as e.g. catheters, stents, balloons and grafts, in the form of mixtures with polymers, solutions, dispersions, and gels that contain at least one cellulose compound and the active ingredients can exist in the forms of free molecules or ions, micelles, liposomes, or microparticles or nanoparticles that can be suitably coated. In the most preferred embodiment the coating system of the present invention comprising at least one active ingredient together with a coating containing at least one cellulose compound is coated on balloons or stents.

The active pharmaceutical ingredient can be any substance intended to treat a disease including any active pharmaceutical ingredient that can prevent or treat restenosis including but not limited to antineoplasmatic, antimicrotubular, antiproliferative, antiinflamatory, immunosuppressive drugs. In a preferred embodiment the antistenotic agents are retinoic acid receptor ligands, for example retinoic acid. In yet one preferred embodiment the antistenotic agents are antimicrotubular and/or immunosuppressing drugs including as examples but not limited to paclitaxel, sirolimus, tacrolimus, everolimus.

In a preferred embodiment the medical device is a biodegradable or non-biodegradable stent made of metal alloy such as stainless steel or cobalt chromium alloy or magnesium or zinc or aluminum alloy, or any other alloy or made of biodegradable or non-biodegradable polymers such as polylactic acid or polyglycolic acid or polycaprocactone or other polymers and their copolymers and mixtures. The above materials are mentioned here as examples and do not limit the application of the invention.

In another preferred embodiment the medical device is a balloon. In yet a more preferred embodiment the balloon is an endovascular balloon, coronary or peripheral. The above devices are mentioned here as examples and do not limit the application of the invention.

In yet other preferred embodiments the medical device is an implant or graft, or patch. The above devices are mentioned here as examples and do not limit the application of the invention.

According to another embodiment of the present invention, a process for the preparation of a coating system for medical devices for the prevention and/or treatment restenosis and neointimal formation during or after vascular procedures and/or implantation of implants comprising an implantable and non-implantable device, such as stent, balloon or graft, wherein said coating system comprising at least one cellulose compound, and therapeutically effective amount of at least one active pharmaceutical ingredient, said process comprises the following steps:
Preparation of a first mixture of at least one cellulose compound and dilution or dispersion of said cellulose compound in an appropriate solvent;
Preparation of a second mixture of an active pharmaceutical ingredient in an appropriate solvent;
Application of the first and the second mixtures each one successively or mixed together on the surface of the device such as a stent, balloon or graft with a suitable method, including but not limited to dipping, immersion, spraying, preferably microspraying, casting in one or more steps, in a single or successive layers; and
Removal of possible remaining solvents with evaporation.

In a preferred embodiment, medical devices are coated with a mixture or solution containing at least one cellulose compound or derivative thereof. In another preferred embodiment the coating system additionally comprises at least one additional compound intended to modify or adjust the coating properties and/or at least one additional compound intended to treat or prevent a disease or pathological condition. The coating solution is applied on the device with any suitable method including as examples dipping, immersion, spray coating, micropipetting, vapor condensation.

The solvents used for the preparation of the coating solution mentioned above may be selected from solvents with various polarities, said solvents being inorganic or organic solvents or supercritical fluids or mixtures thereof alone or in combination with each other and/or with additional compounds. Inorganic solvents according to the present invention may be selected from any aqueous or non aqueous solvent or supercritical fluid including as examples but not limited to ammonia, carbon dioxide, carbon disulfide, carbon tetrachloride, hydrogen fluoride, phosphorus tribromide, sulfuric acid, sulfuryl chloride fluoride, supercritical carbon dioxide, water. Organic solvents according to the present invention may be selected from any organic solvent alone or in mixture, including as examples but not limited to acetaldehyde, acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethyl ether, diglyme (diethylene glycol, dimethyl ether), 1,2-dimethoxy-, ethane (glyme, DME), dimethyl-, formamide (DMF), dimethyl sulfoxide (DMSO), 1,4-dioxane, ethanol, ethyl acetate, ethylene glycol, glycerin, heptane, Hexamethylphosphoramide, (HMPA), Hexamethylphosphorous, triamide (HMPT), hexane, methanol, methyl t-butyl, ether (MTBE), methylene chloride, N-methyl-2-pyrrolidinone, (NMP), nitromethane, pentane, Petroleum ether (ligroine), 1-propanol, 2-propanol, pyridine, tetrahydrofuran (THF), toluene, triethyl amine, o-xylene, m-xylene, p-xylene.

In a preferred embodiment of the present invention the solvent is organic and is preferably selected of a group consisting of ethanol, ethyl acetate and chloroform alone or in mixture. In another preferred embodiment of the invention the solvent is a mixture of organic and inorganic solvents containing by volume between 10 and 90% ethanol, between 10 and 90% ethyl acetate and between 0.1 and 50% water.

In a preferred embodiment of the present invention the cellulose compound is hydroxypropyl cellulose, the solvent used for the preparation of the solution is ethanol and the coating solution contains the cellulose compound in a concentration by weight between 0.1% and 30% and preferably between 0.5% and 5%.

In yet another preferred embodiment the coating solution contains hydroxypropyl cellulose between 0.1% and 30% and preferably between 0.5% and 5% by weight and an active pharmaceutical ingredient (API) at a mass ratio of cellulose compound:API between 100:1 and 1:100, preferably between 100:10 and 10:100, said API preferably being selected from a group containing paclitaxel, sirolimus and its derivatives and retinoic acid receptor ligands. In a further preferred embodiment the medical device is an endovascular stent or endovascular balloon and the coating mixture is applied on the surface of the device with ultrasound spraying or micropipetting.

In another preferred embodiment of the present invention the cellulose compound is ethyl cellulose, the solvent used for preparation of the solution is selected from ethanol, ethyl acetate and their mixtures in various proportions with or without water. The coating solution contains said cellulose compound in a concentration by weight between 0.1% and 30% and preferably between 0.5% and 5%.

In yet another preferred embodiment the coating solution contains ethyl cellulose between 0.1% and 30% and preferably between 0.5% and 5% by weight and an active pharmaceutical ingredient (API) at a mass ratio of cellulose compound:API between 100:1 and 1:100, preferably between 100:10 and 10:100, said API preferably being selected from a group containing paclitaxel, sirolimus and its derivatives and retinoic acid receptor ligands. In a further preferred embodiment the medical device is an endovascular stent or endovascular balloon and the coating mixture is applied on the surface of the device with ultrasound spraying or micropipetting.

Coatings made of cellulose compounds of the present invention including ethyl cellulose, hydroxypropyl cellulose, carboxyhydroxypropyl cellulose, hydroxylpropyl methyl cellulose, methyl ethyl cellulose, methyl cellulose carboxymethyl cellulose and sodium carboxymethyl cellulose have been tested for biocompatibility with the methods described in ISO 10993 and found to be biocompatible and hemocompatible.

Additionally selected cellulose compounds have been tested for possible effects on the proliferation of human arterial smooth muscle cells (HASMCs). The cells were cultured alone or together with cellulose compounds including ethyl cellulose, hydroxypropyl cellulose, carboxyhydroxypropyl cellulose, hydroxylpropyl methyl cellulose, methyl ethyl cellulose, methyl cellulose carboxymethyl cellulose and sodium carboxymethyl cellulose. The tested compounds were added to proliferative HCASMCs cultures in the log phase of cell growth at a range of concentrations similar to that expected to be caused by a coated medical device and cell proliferation was determined at regular intervals up to 12 days after the start of the experiment by cell counting. The tested excipients did not have any unspecific cytotoxic and/or apoptotic effects on HCASMCs when added alone in the tested concentrations and conditions.

The following examples illustrate preferred embodiments in accordance with the present invention without limiting the scope or spirit of the invention:

### EXAMPLES

### Example 1: Coating System for Vascular Stents

A coating solution was prepared by dissolving 4 g of hydroxypropyl cellulose in 100 mL ethanol. The obtained solution was applied on the surface of endovascular stents with 2.5 mm diameter and 30 mm length by an automated dipping process in multiple steps and the excess of solvent was left to evaporate.

The coating integrity on the stents was tested before and after multiple repeats of expansion and re-crimping of the stent and was found to be intact, thus having a favorable mechanical behavior.

Stents made of a magnesium alloy were coated with the coating system of Example 1. The stents were tested in a blood flow simulator with the use of simulated body fluid in comparison with similar uncoated stents as controls. The integrity of the stents was recorded regularly with an optical system for a period of 5 days. At the end of the 5 day period, the coated stents showed better integrity and less fractures than uncoated stents, thus suggesting favorable properties of the coating for bioprotection, i.e. protecting the integrity of the device and delaying corrosion due to contact with the simulated body fluid.

Furthermore, stainless steel stents prepared according to the method of Example 1 were implanted in the iliac arteries of rabbits for 28 days. The animals were euthanized and the stented segments underwent histology examination in comparison with similar control uncoated stents. The results showed less inflammation and similar rate of stenosis compared with controls, suggesting favorable biocompatibility of the coated stents versus the uncoated.

### Example 2: Coating System for Vascular Balloons

A coating solution was prepared by dissolving 1 g of ethyl cellulose and 1 g of paclitaxel in 100 mL ethyl acetate solution. The obtained solution was applied on the surface of an endovascular balloon with the use of an ultrasound spray coater and the excess of solvent was left to evaporate.

Vascular balloons with diameter 2.5 mm and 30 mm length were coated with the above mixture, the paclitaxel mass being 3.5 micrograms per square millimeter of balloon surface.

The balloons were tested in a rabbit iliac artery model for 28 days and showed favorable results in terms of low stenosis rate and preservation of the arterial lumen, as shown by histomorphometric analysis, compared with control uncoated balloons and with existing commercial drug eluting balloons containing the same drug load.

### Example 3: Coating System for Endovascular Balloons

A coating solution was prepared by dissolving 0.5 g of ethyl cellulose, 0.5 g of hydroxypropylcellulose and 1 g of everolimus in 100 mL solution of ethanol and ethyl acetate in a 50:50 mixture by volume. The solution was applied on the surface of an endovascular balloon with the use of an ultrasound spray coater and the excess of solvent was left to evaporate.

Vascular balloons with diameter 2.5 mm and 30 mm length were coated according to the method of example 3, the everolimus mass being 3.0 micrograms per square millimeter of balloon surface.

The balloons were tested in a rabbit iliac artery model for 28 days and showed favorable results in terms of low stenosis rate and preservation of the arterial lumen, as shown by histomorphometric analysis, compared with control uncoated balloons.

Example 4: coating system for endovascular stents A coating solution was prepared by dissolving 1 g of ethyl cellulose and 0.25 g of all trans-retinoic acid in 100 mL ethanol solution. The solution was applied on the surface of an endovascular stent with the use of an ultrasound spray coater and the excess of solvent was left to evaporate.

Vascular coronary stents with diameter 2.5 mm and 10 mm length were coated with the obtained solution, and the retinoic acid mass being 1.0 micrograms per square millimeter of stent surface.

The stents were tested in a rabbit iliac artery model by implantation for 28 days. Histomorphometric analysis of the stented artery after 28 days showed favorable results in terms of low stenosis rate and preservation of the arterial lumen, compared with control uncoated stents.

The present invention has specific advantages compared to the prior art. These advantages include use of sustainable materials, effectiveness and safety, economy in materials and production and an improved production method.

It has been surprisingly found that cellulose compounds used as coatings on medical devices are biocompatible and possess favorable chemical, biochemical and mechanical properties and are suitable for example for drug elution, improvement of biocompatibility, improvement or change of the surface lubricity and other properties as well as for protecting the device from contact with the body tissues and fluids.

While the present invention has been described with respect to the particular embodiments, it will be apparent to those skilled in the art that various changes and modifications may be made in the invention without departing from the spirit and scope thereof, as defined in the appended claims.

What is claimed is:

1. A coating system for a medical device comprising at least one layer of a cellulose compound or derivative thereof applied on a surface of the medical device.

2. The coating system according to claim 1, wherein the coating system further comprises at least one active ingredient for the prevention and treatment of any disease such as vascular disease and restenosis, bone, dermal, neoplastic or any other disease.

3. The coating system according to claim 1, wherein the cellulose compound is selected form microcrystalline cellulose, processed celluloses, hydroxyethylcellulose, cellulose acetate phthalate, hydroxypropylmethyl cellulose phthalate, various esters of cellulose, such as celluloses acetate, triacetate, propionate, sulphate, nitrate, various cellulose ethers (alkyl celluloses), such as ethyl cellulose, hydroxypropyl cellulose ethyl-methylcellulose, carboxymethylcellulose, benzyl cellulose, hydroxyethylcellulose, carboxycellulose derivatives, such as 2,3-Dicarboxycellulose, 6-Carboxycellulose, cellulose acetals, such as formaldehyde and acetaldehyde acetals, aminocellulose, cationic cellulose derivatives and any other type of cellulose or cellulose derivative.

4. The coating system according to claim 2, wherein the at least one active ingredient is any active ingredient selected from lipophilic, hydrophilic, amphiphilic drugs, antiprolifereatives, antineoplastics, antiinfectives, antiviral agents, anticoagulants, peptides and proteins, nucleic acids, steroids, cellular receptor ligands and mixtures thereof.

5. The coating system according to claim 1, wherein the medical device is an implantable or non-implantable medical device, such as stent, balloon or graft, and wherein said coating system is applied on thereon.

6. The coating system according to claim 2, wherein the cellulose compound is a cellulose ether such as ethyl cellulose and the active ingredient is a cytostatic or antiproliferative drug, such as paclitaxel, rapamycin and retinoid receptor ligands and analogues thereof.

7. The coating system according to claim 2, wherein the cellulose compound is a cellulose ether such as hydroxypropyl cellulose and the active ingredient is a cytostatic or antiproliferative drug, such as paclitaxel, rapamycin and retinoid receptor ligands, and analogues thereof.

8. The coating system according to any claim 1, wherein said coating system is applied on the surface of the medical device for the prevention and treatment of vascular disease and restenosis, by dipping, immersion, micropipetting, spraying, preferably microspraying or ultrasound spraying.

9. The coating system according to claim 1, wherein said medical device is a medical balloon such as an endovascular coronary or peripheral or special purpose balloon comprising ethylcellulose or hydroxypropyl cellulose as coating at a mass density of about 0.1 to 10 micrograms per square millimetre preferably between 0.5 and 5 micrograms per square millimetre and a therapeutically effective amount of an active ingredient, such as paclitaxel, sirolimus, retinoid receptor ligands or analogues thereof, at a mass ratio of ethylcellulose or hydroxypropyl cellulose versus drug of about 0.01 to 20 and preferably between 0.1 to 2.

10. The coating system according to claim 1, wherein said medical device is a vascular, coronary or peripheral or renal stent, that is made of cobalt chromium, or stainless steel, or a memory-responsive metal, or other metal alloy, or a polymer or a blend of polymers comprising ethylcellulose or hydroxypropyl cellulose as coating at a mass density of about 0.1 to 10 micrograms per square millimetre and a therapeutically effective amount of an active ingredient, such as paclitaxel, sirolimus, retinoid receptor ligands or analogues thereof, at a mass ratio of ethylcellulose or hydroxypropyl cellulose versus drug of about 0.01 to 20 and preferably between 0.1 to 2.

11. A process for the preparation of a coating system for a medical device for the prevention and/or treatment restenosis and neointimal formation during or after vascular procedures and/or implantation of implants comprising an implantable and non-implantable device, such as stent, balloon or graft, wherein said coating system comprising at least one cellulose compound or derivative thereof, and a therapeutically effective amount of at least one active ingredient, said process comprising the following steps:
Preparation of a first mixture of at least one cellulose compound and dilution or dispersion of said cellulose compound in an appropriate solvent;
Preparation of a second mixture of an active ingredient in an appropriate solvent;
Application of the first and second mixtures on a surface of the medical device such as a stent, balloon or graft with a suitable method, selected from dipping, immersion spraying, preferably microspraying, casting in one or more steps, in a single or successive layers; and
Removal of possible remaining solvents with evaporation.
